(19) United States
(12) Patent Application Publication
(10) Pub. No.: US 2022/0233123 A1
(43) Pub. Date: Jul. 28, 2022

(54) Method and Apparatus for Motion Dampening for Biosignal Sensing and Influencing

(71) Applicant: Sens.ai Inc., Whistler (CA)

(72) Inventors: Paola Telfer, Whistler (CA); Corey Julihn, Surrey (CA)

(21) Appl. No.: 17/618,351

(22) PCT Filed: Jun. 10, 2020
(86) PCT No.: PCT/IB2020/055469

§ 371 Date: Dec. 10, 2021

Related U.S. Application Data
(60) Provisional application No. 62/859,698, filed on Jun. 10, 2019.

(51) Int. Cl. A61B 5/256; A61B 5/30; A61B 5/263; A61N 1/04; A61N 1/20; A61N 2/00; A61N 5/06; A61B 5/024; A61B 5/245; A61B 5/28; A61B 5/291; A61B 5/296; G06F 3/01

(52) U.S. Cl. CPC A61B 5/256; A61B 2562/164; A61B 5/263; A61N 1/0456; A61N 1/0472; A61N 1/20; A61N 2/006; A61N 5/0622; A61B 5/02416; A61B 5/245; A61B 5/28; A61B 5/291; A61B 5/296; G06F 3/015; A61B 5/30

(57) ABSTRACT

Devices and methods for electrical potential sensing and influencing are provided. The inventive devices include electroencephalography (EEG), electrocardiogram (EKG), photoplethysmography (PPG), electromyography (EMG), and temperature devices for measuring bio-activity signals from a body. The described devices are designed to include motion dampending, a hybrid non-contact and contact sensing surface and to optimise sensitivity in difficult sensing conditions, such as during movement, through obstructions like hair and clothing, while having a convenient and small form factor. The inventive devices provide for improved sensitivity, adaptability, and noise reduction when compared to other designs. Methods for influencing said bio signals with a device with a hybrid non-contact and contact sensing surface are also described.

### FIELD OF THE INVENTION

The present invention relates to devices and methods for motion dampening for biosignal sensing and influencing. More specifically, the devices are designed to place non-contact and/or contact sensing surfaces within an electric or biometric field generated by a subject and to optimise sensitivity and reduce noise in difficult sensing conditions, such as when a subject is moving and through obstructions like hair. The invention also relates to methods for obtaining and influencing said biosignals.

### BACKGROUND OF THE INVENTION

Bioelectric sensors such as Electroencephalogram (EEG) and electrocardiogram (ECG or EKG) sensors measure the electric fields of the brain and heart. Most commercially available EEG and ECG sensors rely on the provisioning of direct electrical contact with the skin. When the sensing location on the skin is obstructed, for example with hair, conductive gel is often used to overcome the lack of direct electrical contact. Another common approach is the use of dry brush electrodes which penetrate between the hair and require pressure on the contact point which can be uncomfortable or painful. A key challenge faced by biosensing devices is that the targeted signal is often polluted with noise. Sources of noise can include other bioelectric signals such as EMG (muscle/motor neurons), noise inherent in the electronics, movement of subject and therefore the sensing surfaces, and external electromagnetic fields including radio waves. More specifically, EEG signals are very small and typically range from 10 uV to 100 uV and are therefore highly sensitive to noise.

More recently, non-contact electric potential sensors have been developed. These non-contact sensors rely on capacitive coupling between the skin and a sensing plate. These sensors have successfully demonstrated non-contact sensing of EEG and ECG signals, but have still had limited success in sensing through obstructions such as hair. These sensors still commonly suffer from interference from the aforementioned sources of noise and experience poor signals in real-world obstruction situations where the amount of obstructing material (i.e. hair) varies across multiple sensing locations, wearers, and over time.

Non-contact sensor designs rely on a flat ridge sensing plate for capacitive coupling. Examples can be found in U.S. Pat. No. 8,694,084, Harland 2001, Oehler 2008, Portelli 2017, Chi 2009 and Chi 2010. These non-contact sensing plates suffer from weak coupling between the electrode and body due to obstructions and other issues. In order to overcome this, the sensing plates are made larger in an effort to increase signal-to-noise ratio (SNR). This can often involve increasing the size of the detection disc to approximately double the diameter of typical wet electrodes (Portelli, 2017). The sensing plates shown in the '084 patent incorporate insulation that means that they can only operate in non-contact mode.

Earlier examples of non-contact EEG and EKG sensing can be found, for example in U.S. Pat. No. 5,473,244, but only non-contact methodologies are shown, with the known drawbacks associated with signal strength and low SNR. More recently, non-contact sensing methodologies have been applied to the sensing of physiological states such as drowsiness, but they do not obviously overcome the aforementioned drawbacks of non-contact devices and methodologies.

Influencing biometric signals from the body crosses many disciplines and methods including medication, therapy, meditation, breathing exercises, biofeedback, neurofeedback and biostimulation. Neurostimulation is one form of biostimulation which involves the purposeful modulation of nervous system activity. Photobiomodulation (PBM) uses modulating near-infrared light and can be applied to stimulate the nervous system.

Precise placement of biosensors and biostimulators is important when sensing and influencing biological signals. Devices are typically designed to place sensors and stimulators firmly against the skin or as close as possible to the targeted electric field in the case of non-contact sensors. A key challenge for sensor placement is that subjects vary in size and shape and introduce motion artifacts due to voluntary and involuntary movements, such as breathing, blinking, swallowing and head movement. One approach used by devices sensing EEG signals is to use a semi-ridge adjustable band which wraps around the subject's head and has one or more flexible arms with sensors on them such as described in U.S. Pat. No. 8,706,182, 20170332964A1, 20180092599A1, 20160316288A1. This well known approach generally accomplishes the task of sensor placement, however, this approach requires a firmer fit then the present invention and is therefore less comfortable for the subject. Another drawback to this approach is that each sensor usually requires an additional arm for placement, otherwise differences in body sizes and shapes alter the quality of the sensor placement. Additionally, the further the flexible arms travel from the hub or main band(s) the more they are likely the sensors will move due to motion of the subject.

Another approach often used by EEG and EKG devices is to place sensors using a flexible wrap or cap often made of fabric which is secured by an elastic or fabric strap; one such example can be seen in U.S. Pat. No. 9,668,694B2. A downside to this approach is that different head or body shapes will vary the pressure of the sensors across different areas of the body. Additionally when sensors are required on more then one axis, this approach usually requires devices to be made in multiple sizes. Finally, these flexible caps are typically secured with a strap which can introduce motion artifacts. For example many EEG caps are secured with a strap passing along the chin, where jaw movements (i.e. swallowing or talking) will be translated into the cap and sensors, introducing noise into the signal.

The current inventors seek to address the deficiencies of biometric sensing and influencing devices by providing a design that places sensors and stimulators in the correct locations, adjusts to different subject shapes and sizes, reduces signal noise from motion, provides consistent sensor pressure across multiple axises, increases user comfort, recovers from displaced or moved sensors, and increases the electrical coupling between sensing and stimulating surfaces and the targeted biometric fields.

### SUMMARY OF THE INVENTION

Accordingly, the present invention relates to a wearable device which includes two or more semi-flexible bands where said bands position one or more flexible membranes against a body and said devices may include a biosensor system and may include a biostimulation system. The present invention is capable of accurately placing sensors in targeted locations with consistent pressure across multiple axises, adapting to different body shapes and sizes, limiting effects of motion artifacts, recovering from displaced or moved sensors, and increasing the electrical coupling between the sensing surfaces and the target electric fields or other target biometric fields.

Additionally the present invention relates to a biosensor electrode for sensing electric fields from a body which is soft, flexible, elastic, and non-flat. This sensing surface may provide capacitive coupling or direct coupling to the targeted electric field. Conforming the sensing surface to the shape of the body increases the sensing surface area placed within the electric field increasing the effect of capacitive coupling. This sensing surface limits motion and recovers from displacement by utilizing its elastic force to dampen motion and fill air gaps. Additionally, these sensors may be more comfortable and do not require the sensing surface to be forced down onto the body with excessive pressure, and are not abrasive.

The present invention further relates to methods for influencing biosignals from one or more subjects. Where biosignals from the body are processed, analyzed and used to provide feedback to the subject. Wherein analyzing the biosignals relates to assessing the subject's mental, physiological, psychological, somatic and/or autonomic health and/or states and the feedback is intended to help the subject adjust or change said analyzed health and/or states. Feedback to the subject may include audio, visual, vibration, haptic, movement or changes in another object or device, or other means of sensory feedback and further include biostimulation feedback such as Photobiomodulation (PBM). Further forms of feedback may include information, recommendations, diagnosis, or instructions via text, audio, or other means.

### BRIEF DESCRIPTION OF THE DRAWINGS

The invention will be better understood according to the following detailed description of several embodiments with reference to the attached drawings, in which:

FIG. 1 presents a perspective view of an exemplary device capable of accurately and comfortably placing biosensors and biostimulators on different head shapes and sizes, according to one embodiment of the present invention.

FIG. 2 shows a side view of an exemplary device capable of accurately and comfortably placing biosensors and biostimulators on different head shapes and sizes, according to one embodiment of the present invention.

FIG. 3 shows a top down view of an exemplary device capable of accurately and comfortably placing biosensors and biostimulators on different head shapes and sizes, according to one embodiment of the present invention.

FIG. 4 displays a top down view of an exemplary device with multiple membranes and another with webbed membranes, according to one embodiment of the present invention.

FIG. 5 presents an exemplary sensor system with a soft, flexible, elastic, and non-flat sensing surface, including bump features extending from the core, according to an embodiment of the present invention.

FIG. 6 shows a sensor system according to an embodiment of the present invention with a guard shield to prevent outside electrical interference.

FIG. 7 presents a sensor system with a soft, flexible, elastic, and non-flat sensing surface as it conforms to the shape of a body when compressed.

FIG. 8 presents an alternative sensor system according to an embodiment of the present invention where the soft, flexible, elastic, and non-flat sensing surface, has no additional features extending from the core.

FIG. 9 presents a flow chart of a sensing protocol methodology according to an embodiment of the present invention.

### DETAILED DESCRIPTION OF THE INVENTION

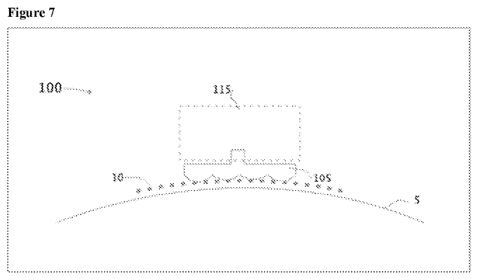

The present invention relates to a biosensor electrode for sensing electric fields from a body comprising:
a soft, flexible, elastic, and non-flat electrode core which may include one or more features extending from its outermost surface.
   wherein the total height of the electrode core and features must be at least 10% the width or length of the core, whichever is greater.
   wherein the durometer of the electrode core and features must be less than 50 Shore A and ideally less than 10 Shore A.
   wherein features extending from the electrode core surface must be less than 50% of the total height of the electrode
   wherein the surface area of features when compressed against both a sphere with a circumference of 55 centimeters and flat surface with a force of 250 grams must comprise at least 30% of the surface area of the outermost surface of the electrode core.
   a sensing surface or conductive coating on said electrode core and extending features
   An electrical connection from the sensing surface to an amplifier
      wherein the biosensor surface conforms to the shape of the subject's body. This sensing surface may provide capacitive coupling or direct coupling to the targeted electric field. Conforming the sensing surface to the shape of the body (FIG. 7) increases the sensing surface area placed within the electric field increasing the effect of capacitive coupling. Further said sensing surface limits motion and recovers from displacement by utilizing its elastic force to dampen motion and fill air gaps between the surface and the subject's body.

In one embodiment the conductive coating consists of a conductive fabric, which may include silver, nickel, copper, gold, graphene, and/or other conductive coatings. In another embodiment the conductive coating may consists of a flexible coating of graphene or a flexible silicone or polymer embedded or coated with a conductive material such as silver, nickel, copper, gold, silver nanowire, and/or carbon nanotubes

In one aspect, the present invention includes a capacitive biosensor system utilizing a hybrid contact and non-contact sensing surface. The sensing surface in the present invention is non-flat providing a number of advantages over prior art including pushing aside or through obstructions such as hair or clothing, reduced overall size while maintaining an increased capacitive coupling through increased surface area, the ability to be placed on the body with less pressure, and the ability to work in both contact and non-contact modes.

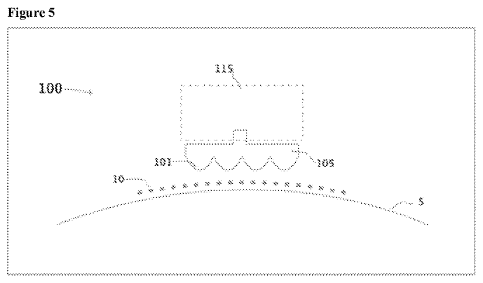

In one embodiment the features extending from the electrode core may include spherical bumps, prongs, ridges, protruding rings, facets, or other extrusions from the base of the surface (FIG. 5). This shape may be optimized to the application; where an ideal surface balances:
maximizing surface area near the body, thus increasing the capacitive effect
passing through obstructions and placing as much of the sensing surface as close to the source of electric field as possible, reducing the air gap and obstructions increases the capacitive effect
making contact with the body, thus creating an opportunity for direct coupling
comfort of the subject

Additionally, the overall size of the sensing surface may be adapted based on the application where increasing the size increases the capacitive coupling capacity.

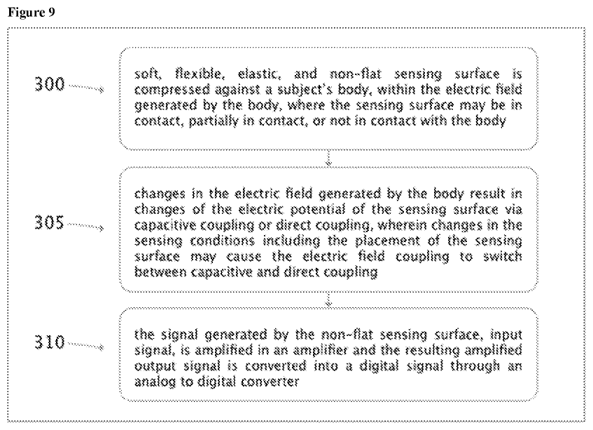

The soft, flexible, elastic and non-flat sensing surface is compressed against a body, and placed within the electric field generated by the body. The surface may be in contact, partially in contact, or not in contact with the body. The placement may be subject to changing non-ideal conditions including obstructions, hair or body products, body oils, movement, displacement, and varying degrees of contact with the body surface. Changes in the electric field generated by the body result in changes of the electric potential of the sensing surface via capacitive coupling and/or direct coupling. The signal generated by the sensing surfaced due to changes in the body's electric field is amplified and converted into a digital signal and sent to a computer, phone, wearable, server and/or other device through wired or wireless connection such as Bluetooth, WiFI, cellular, or internet where is may be processed, stored, displayed, and/or interpreted (FIG. 9).

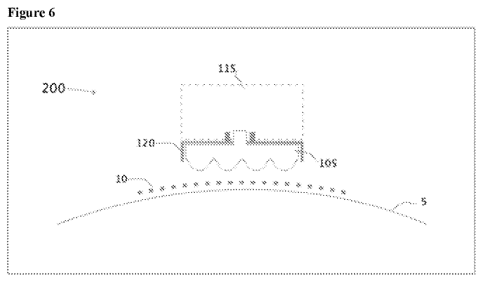

In yet another embodiment the present invention may incorporate a guard shield which limits the pickup of electric fields from other sources (FIG. 6). There are various methods for shielding electrodes; in one technique the shield, being made of conductive material such as copper, is driven with a signal matching the input voltage from the capacitive sensor.

In a preferred embodiment one or more biosensors are placed in a wearable device such as a headset, and placed on the body.

The present invention relates to a device for capturing and/or influencing biosignals from a subject comprising:
Two or more semi-flexible or rigid anchors.
Two or more semi-flexible bands, each band having two ends, where at least one end is connected to at least one anchor. Wherein semi-flexible bands follow the curvature of the subject's body and form an opening between said bands.
One or more flexible membranes. Each flexible membrane connects to at least two bands at a least one point respectively.
Each membrane and anchor containing zero or more biosensors
Each membrane and anchor containing zero or more biostimulators.
Containing at least one or more biosensors or biostimulators.
   wherein placing the anchors in the correct location of the subject's body, adjusting the size of the anchors and/or bands places the biosensors and biostimulators within the targeted areas of the body. When in place the flexible membrane stretches, flexes and conforms to the shape of the subject's body, and is held in tension by the semi-flexible bands and anchors. Wherein connecting said membranes to more then one said band allows the membrane to distribute pressure evenly along multiple axises, as opposed to just the axis long which a single band runs. Wherein at least two anchors touch the subject's body, applying a force toward the body. In an ideal embodiment the force of the anchors is created by the elastic force of the semi-flexible bands connecting to the anchors.

In one embodiment, the device contains embedded biosensors located in the flexible membranes, and/or anchors, with their sensing surface extending outward toward the subject. In the preferred embodiment the embedded biosensors are, as described previously, soft, flexible, elastic and non-flat sensing surfaces which conform to the shape of the subject's body, thus increasing the surface area that is placed within the electrical field generated by the body. Obtained signals are amplified and may be sent to a computer, phone or wearable device. The signals may be displayed, stored and/or processed.

In one embodiment the device includes biostimulators located in the flexible membranes, and/or anchors, with their stimulation surface extending outward toward the subject. The preferred embodiment utilizes non-invasive Photobiomodulation (PBM) stimulation. PBM therapy is the use of non-ionizing photonic energy to create photochemical changes inside cellular structures usually mitochondria. Other embodiments may include PEMF (Pulsed Electromagnetic Field), tMS (Transcranial magnetic stimulation), tACS (Transcranial Alternating Current Stimulation), tRNS (Transcranial Random Noise Stimulation), tDCS (Transcranial Direct Current Stimulation).

In one embodiment the anchors may include a known mechanism to adjust their length, either to increase or decrease the length of said anchor, thus allowing the device to adapt to different body sizes. In another embodiment the semi-flexible bands may include a known mechanism to adjust their length, either to increase or decrease the length of said band, where the bands may be individually adjusted, thus allowing the device to adapt to different body sizes.

In one embodiment the anchors include one or more hinges, enabling the device to be folded into a more compact form for storage. In another embodiment at least one band includes one or more hinges, enabling the device to be folded into a more compact form for storage.

In a preferred embodiment the bands run across the body in the same direction, In other embodiments the bands may cross each other or connect to each other.

In a preferred embodiment, the membrane(s) run between the bands, bridging across the openings. The membrane(s) may be filled-in covering the area of the body, separate bands running across the body, mesh, webbed, or another shape. In another embodiment the membranes can be made of a soft flexible rubber, silicone, a flexible textile, or another soft flexible material. In an ideal embodiment the membrane has a durometer of less than 40 Shore A.

In one embodiment said biosensors consist of at least one ground electrode, and at least two signal acquisition electrodes, where at least one signal acquisition electrode is used as a reference electrode for at least one other signal electrode.

In one embodiment said biosensors comprise at least one non-contact electric potential sensor. In another embodiment said biosensors comprise at least one contact electric potential sensor. In other embodiments, said biosensors comprise at least one of photoplethysmography (PPG) sensor, Functional near-infrared spectroscopy (fNIRS) sensor, magnetoencephalography (MEG) sensor. In another embodiment said biosensors comprise at least one skin conductivity sensor. In yet another embodiment said biosensors comprise at least one temperature sensor.

In one embodiment said biosensors are configured to capture EEG signals, and or EKG or ECG signals and or EMG (electromyography) signals. Wherein said biosensors are connected to an amplifier, one or more passive filters, an analog digital converter and optionally a wireless transmitter and receiver.

In another embodiment the device comprises at least one speaker. One iteration of this embodiment comprises speakers embedded in the anchors of the device wherein the anchors are an embodiment of headphone speakers. In yet another embodiment the device is embedded within a hat or a helmet. In another embodiment the device is embedded within a Virtual Reality headset, or an Augmented Reality headset, or another sensory augmentation device. In yet another embodiment, the device is used as a Brain Computer Interface (BCI).

In an optional embodiment said device consists of:
Two or more semi-flexible or rigid anchors.
Two or more semi-flexible bands, each band having two ends, where at least one end is connected to at least one anchor. Wherein semi-flexible bands follow the curvature of the subject's body and form an opening between said bands.
One or more flexible membranes. Each flexible membrane connects to at least two bands at a least one point respectively.
Each membrane and anchor containing zero or more biosensors
Each membrane and anchor containing zero or more biostimulators.
Containing at least one or more biosensors or biostimulators.
   wherein said membrane consists of another known sensor placement device. In one such embodiment said membrane is an EEG cap attached to said bands and the resulting EEG cap does not require a strap around the chin or head.

The present invention relates to methods for influencing biosignals from one or more subjects, the method comprising the following steps:
Placing the aforementioned device(s) for capturing and/or influencing biosignals from a subject, on each subject's body
Using the device(s) to acquire biosignals from each subject
Using the device to process and analyze the biosignals or transmitting the signals to another device where the signals are then processed and analyzed.
Using the analyzed signal to provide feedback to the subject(s)
Optionally continuing to acquire, process, analyze biosignals, and provide feedback to the subject(s) in a feedback loop.
   wherein analyzing the biosignals relates to assessing the subject's mental, physiological, psychological, somatic and/or autonomic health and/or states and the feedback is intended to help the subject adjust or change said analyzed health and/or states. Feedback to the subject may be provided in different forms including audio, visual, vibration, haptic, movement or changes in another object or device, or other means of sensory stimulation and additionally includes biostimulation feedback such as Photobiomodulation (PBM). Further forms of feedback can include metrics, information, recommendations, diagnosis, or instructions via text, audio, or other means.

In one embodiment the method for influencing biosignals pertains to one subject wearing said device, and receiving said feedback from the device, wherein no external feedback mechanisms are in place. In another embodiment, the device transmits the acquired biosignals to another device such as a computer or mobile device, where the signal is processed and feedback is provided. In yet another embodiment the device wirelessly transmits the acquired biosignals to a server where the signal is processed and feedback returned through the inventive device, a computer, or a device, or another device.

In one embodiment more then one subjects are each wearing a device, where said biosignals are collectively transmitted to a server for processing and analysis and feedback is provided based on individual and group biosignals.

In yet another embodiment the device processes and transmits biosignals to another processing device such as a server, computer, or mobile device where the signals are analyzed and a report is generated. Wherein the report includes information pertaining to diagnostic metrics and/or health metrics, and the report is provided to the subject or to an expert in a field pertaining to the report.
Biosensor: an electronic device or electronic circuit which is capable of reading a biosignal from a biometric field. Examples include an EEG electrode, a Pulse Oximeter, and ECG electrode, a glucose sensor, and a temperature sensor.
Biostimulator: an electronic device or electronic circuit which is capable of altering, influencing, or changing a biosignal.
Biosignal: a signal which can be continuously monitored from a body which can be an electrical signal or a non-electrical signal.
Biometric Field: an area surrounding the source of a biosignal in which said biosignal can be read using a biosensor. In the case of an electrical biosignal, this is an electric field.

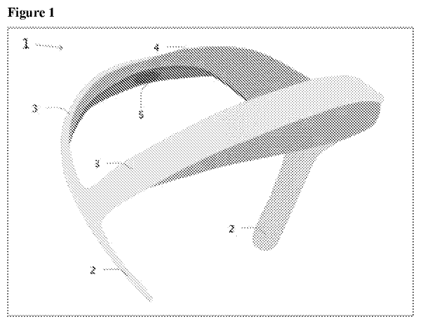

With reference now to the figures, FIG. 1 shows a perspective view of an exemplary device for capturing and influencing biosignals 1 including two anchors 2 with two semi-flexible bands 3 holding in place a flexible membrane 4 with embedded biosensors and/or biostimulators 5.

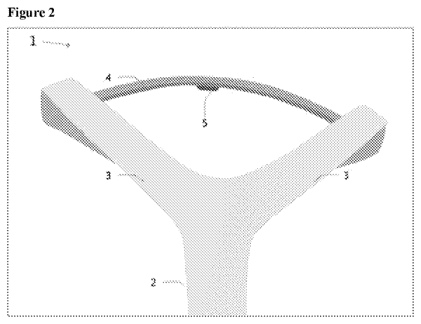

FIG. 2 is a side view of an exemplary device for capturing and influencing biosignals 1 including two anchors 2 with two semi-flexible bands 3 holding in place a flexible membrane 4 with embedded biosensors and/or biostimulators.

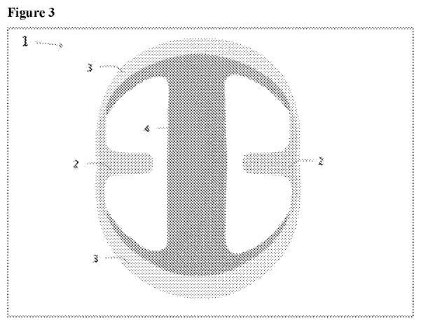

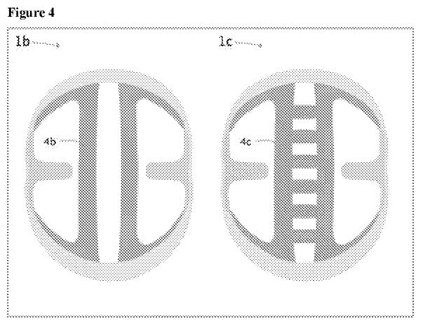

FIG. 3 is a top down view of an exemplary device for capturing and influencing biosignals 1 including two anchors 2 with two semi-flexible bands 3 holding in place a flexible membrane 4 with embedded biosensors and/or biostimulators 5. FIG. 4 provides additional elevation views of an exemplary device for capturing and influencing biosignals.

In FIG. 5, the sensor system 100 includes an elastic non-flat sensing surface 105, with bump features 101 extending from the core, for contact and/or non-contact capacitive coupling to the body 5 through hair or other obstructions 10. The sensing surface 105 is connected to the amplifier 115.

FIG. 6 illustrates an alternative embodiment in which sensor system 200 includes a guard shield 120 around the elastic non-flat sensing surface 105 for contact and/or non-contact capacitive coupling to the body 5 through hair or other obstructions 10. The sensing surface 105 is connected to the amplifier 115.

In FIG. 7, the sensor system 100 includes an elastic non-flat sensing surface 105 for contact and/or non-contact capacitive coupling to the body 5 through hair or other obstructions 10. The sensing surface 105 is connected to the amplifier 115. In this figure the sensing surface 105 is show compressed against and conforming to the shape of the body 5.

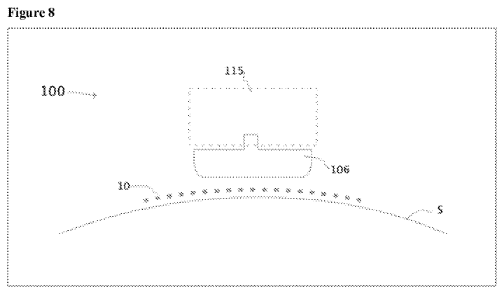

In FIG. 8, the sensor system 100 includes an elastic non-flat sensing surface 105, without additional features extending from the core, for contact and/or non-contact capacitive coupling to the body 5 through hair or other obstructions 10. The sensing surface 105 is connected to the amplifier 115.

FIG. 9 provides a flow chart of a sensing protocol methodology according to an embodiment of the present invention. The protocol starts at 300 where the elastic non-flat sensing surface is placed inside the electric field of a body. In 305 the sensing surface couples to the electric field generated by the body and adapts to changing conditions before 310 being amplified and converted into a digital signal.

As can be understood, the examples described above and illustrated in the figures are intended to be exemplary only. The scope is indicated by the appended claims.

What is claimed is:

1. A biosensor electrode for sensing electrical fields from a body of a user comprising:
a flexible electrode core optionally including one or more features extending from its outermost surface;
a sensing surface disposed on the electric core and features; and
a connection element providing an electrical connection between the sensing surface and an amplifier;
wherein the biosensor surface conforms to the shape of the user's body.

2. The biosensor electrode of claim 1, adapted for use on the user's head and wherein the biosensor surface conforms to the shape of the user's head.

3. The biosensor electrode of claim 1, wherein the biosensor surface provides coupling to a targeted electrical field of the user consisting of capacitive coupling or direct coupling.

4. The biosensor electrode of claim 1, wherein the total height of the features extending from the outermost surface of the electrode core is at least 10% of the [area/width/length] of the electrode core.

5. The biosensor electrode of claim 1, wherein the height of the features extending from the outermost surface are less than 50% of the height of the biosensor electrode.

6. The biosensor electrode of claim 1, wherein the surface area of the features extending from the outermost surface when compressed against both a sphere with a circumference of 55 centimeters and flat surface with a force of 250 grams comprise at least 30% of the surface area of the outermost surface of the electrode core.

7. The biosensor electrode of claim 1, wherein the sensing surface is a conductive coating selected from the group consisting of silver, nickel, copper, gold, graphene conductive fabric, a flexible coating of graphene, a flexible silicone or polymer embedded or coated with a conductive material such as silver, nickel, copper, gold, silver nanowire, and carbon nanotubes.

8. A wearable device for sending and receiving biosignals to and from a user comprising:
at least two anchors;
at least two semi-flexible bands each with a first end and a second end, wherein at least one of the first end and the second end of each of the semi-flexible bands is connected to at least one anchor, and wherein the at least two semi-flexible bands follow the contours of the subject's body such that an opening is formed between the at least two semi-flexible bands;
at least one flexible membrane wherein the at least one flexible membrane is connected to at least two semi-flexible bands;
at least one biosensor optionally disposed on the at least one flexible membrane or one or more of the at least two anchors; and
at least one biostimulator optionally disposed on the at least one flexible membrane or one or more of the at least two anchors;
wherein correct placement of the anchors and correct adjustment of the anchors and semi-flexible bands places the at least one biosensor and at least one biostimulator on a targeted area of the user's body.

9. The wearable device of claim 8 configured to place the at least one biosensor and at least one biostimulator on a targeted area on the user's head.

10. The wearable device of claim 8, wherein the device provides one of PBM stimulation, PEMF stimulation, tMS stimulation, tACS stimulation, tRNS stimulation or tDCS stimulation.

11. The wearable device of claim 8, wherein the at least on biosensor is a PPG sensor, a fNIRS sensor or an MEG sensor.

12. The wearable device of claim 8, wherein the at least one biosensor is configured to capture EEG signals, EKG signals, ECG signals or EMG signals.

13. The wearable device of claim 8, configured to be used as a virtual reality headset, an augmented reality headset or a brain-computer interface.

14. The wearable device of claim 8, wherein at least one biosensor and at least one biostimulator and at least one user's body create a closed feedback loop, wherein the user's body generates a biosignal, the biosignal is captured by a biosensor and a biostimulator influences the user's biosignal.
